(19) United States
(12) Patent Application Publication
(10) Pub. No.: US 2017/0241847 A1
(43) Pub. Date: Aug. 24, 2017

(54) MULTIFUNCTIONAL DISTRIBUTING SENSING AND FABRICS

(71) Applicant: The Regents of the University of California, Oakland, CA (US)

(72) Inventors: Kenneth J. Loh, San Diego, CA (US); Long Wang, La Jolla, CA (US); Sumit Gupta, La Jolla, CA (US); Helen S. Koo, Seoul (KR)

(73) Assignee: The Regents of the University of California, Oakland, CA (US)

(21) Appl. No.: 15/442,569

(22) Filed: Feb. 24, 2017

Related U.S. Application Data
(60) Provisional application No. 62/299,344, filed on Feb. 24, 2016.

(51) Int. Cl. G01L 1/20; A61B 5/053; A61B 5/11; D06M 15/693; A61B 5/00

(52) U.S. Cl. CPC G01L 1/20; D06M 15/693; A61B 5/746; A61B 5/11; A61B 5/7278; A61B 5/7225; A61B 5/0536; G01B 7/20

(57) ABSTRACT

A multi-walled carbon nanotube thin film can be manufactured and operatively attached to a fabric for use with immobilized patients to sense when one or more body parts of an immobilized patient experiences prolonged pressure. A boundary of the multi-walled carbon nanotube thin film can be equipped with a plurality of electrodes that can be injected with current. Voltage measurements can be taken which can be translated into changes in impedance throughout the multi-walled carbon nanotube thin film. The changes in impedance, in turn, are representative of points and magnitudes of pressure experienced by the multi-walled carbon nanotube thin film as a result of the one or more body parts of the immobilized patient contacting and exerting pressure on the multi-walled carbon nanotube thin film.

### CROSS-REFERENCE TO RELATED APPLICATIONS

This application claims priority to, and the benefit of, U.S. Provisional Patent Application Ser. No. 62/299,344 filed on Feb. 24, 2016, which is incorporated herein by reference in its entirety.

### STATEMENT REGARDING FEDERALLY SPONSORED RESEARCH OR DEVELOPMENT

This invention was made with Government support under CMMI-1253564 awarded by the National Science Foundation. The Government has certain rights in the invention.

### TECHNICAL FIELD

The present disclosure relates generally to pressure sensing, and in particular, to distributed/spatial pressure sensing using electrical impedance tomography to estimate the resistivity distribution of a pressure-sensitive medium(s) or material(s), such as fabric, and changes thereto pursuant to applied pressure at different locations on the pressure-sensitive medium(s).

### DESCRIPTION OF THE RELATED ART

Immobilized patients can suffer from sitting, lying, and/or otherwise remaining in one position for prolonged periods of time. Remaining in one position for too long can result in sustained pressure being applied to one or more parts of a patient's body that are in contact with a bed or chair, for example. This in turn, can result in painful and sometimes life-threatening conditions affecting the patient's skin, tissue, and/or bones.

One example of tissue damage that commonly occurs as a result of immobilization are pressure ulcers, which are localized injuries to the skin and/or tissue at those parts of the patient's body that are subjected to the sustained pressure. Pressure ulcers are difficult to treat, and thus preventing their occurrence in the first place is preferable.

### BRIEF SUMMARY OF THE DISCLOSURE

In accordance with one embodiment, a method may comprise mixing multi-walled carbon nanotubes in an aqueous solution, dispersing the multi-walled carbon nanotubes in the aqueous solution, and adding latex and water to create a sprayable multi-walled carbon nanotube film. The method may further comprise depositing the multi-walled carbon nanotube film on a fabric transfer, drying the multi-walled carbon nanotube film, and attaching boundary electrodes about a boundary of the multi-walled carbon nanotube film. The multi-walled carbon nanotube film and the boundary electrodes can be transferred to a fabric, and an adhesive covering can be applied over the multi-walled carbon nanotube film.

In some embodiments, the aqueous solution comprises a 2 weight percent poly (sodium 4-styrenesulfonate) and N-methyl 2-pyrrolindinone aqueous solution. The dispersing of the multi-walled carbon nanotubes may comprise high-energy sonication.

In some embodiments, the water may be deionized water. Transferring the multi-walled carbon nanotube film to the fabric may be accomplished by ironing the multi-walled carbon nanotube film onto the fabric.

In accordance with another embodiment, a method may comprise generating a pressure map representative of pressure exerted on a sensing fabric, and generating an electrical image corresponding to the pressure map. The electrical image may be analyzed to determine one or more areas indicative of prolonged pressure on one or more parts of a body contacting the sensing fabric. Moreover, an alert can be generated suggesting shifting the body based upon the indication of prolonged pressure.

The method may further comprise injecting current into two of a plurality of boundary electrodes comprising the sensing fabric, and measuring the voltage at remaining ones of the plurality of boundary electrodes.

The method may further comprise repeatedly injecting current into alternative pairs of the plurality of boundary electrodes while repeatedly measuring the voltage at the remaining ones of the plurality of boundary electrodes. The generation of the pressure map can be based upon the voltage measurements at the plurality of boundary electrodes. In some embodiments, the measured voltage is amplified and/or filtered.

In some embodiments, analyzing the electrical image can comprise applying an electrical impedance tomography algorithm to reconstruct an image of the sensing fabric indicating changes in impedance in the sensing fabric due to pressure from the one or more parts of the body on the sensing fabric.

In accordance with yet another embodiment, a system can comprise: a fabric; a multi-walled carbon nanotube thin film operatively attached to the fabric; and a plurality of electrodes operatively attached to a boundary of the multi-walled carbon nanotube thin film, wherein alternating pairs of the plurality of electrodes are injected with current, and voltage measurements are taken at remaining ones of the plurality of electrodes, such that a visual representation of points and magnitudes of pressure on the fabric is presented based upon the voltage measurements.

The multi-walled carbon nanotube thin film may comprise a multi-walled carbon nanotube-latex thin film. The fabric, the multi-walled carbon nanotube thin film, and the plurality of electrodes can be washable.

An adhesive layer may cover the multi-walled carbon nanotube thin film such that the multi-walled carbon nanotube thin film is sandwiched between the fabric and the adhesive layer.

An electrical impedance tomography device can be operatively connected to the plurality of electrodes, wherein the electrical impedance tomography device comprises a current switch that injects the current into the alternating pairs of the plurality of electrodes.

In some embodiments, the electrical impedance tomography device comprises a display that displays the visual representation, where the points and magnitudes of pressure on the fabric reflect changes in impedance in the multi-walled carbon nanotube thin film. In some embodiments, the electrical impedance tomography device comprises a notification component that generates one or more notifications or alarms upon the multi-walled carbon nanotube thin film sensing prolonged pressure thereon.

### BRIEF DESCRIPTION OF THE DRAWINGS

The present disclosure, in accordance with one or more various embodiments, is described in detail with reference to the following figures. The figures are provided for purposes of illustration only and merely depict typical or example embodiments.

FIG. 1 is a flow chart illustrating example operations that can be performed to manufacture a multi-walled carbon nanotube thin film in accordance with various embodiments.

FIG. 2 is a schematic representation of certain ones of the example operations for manufacturing the multi-walled carbon nanotube thin film of FIG. 1.

FIG. 3 illustrates an example multi-walled carbon nanotube thin film.

FIG. 4 illustrates an example electrical impedance tomography device and example component parts thereof operatively connected to an example multi-walled carbon nanotube thin film.

FIG. 5 is a flow chart illustrating example operations that can be performed to generate an electrical image representation of sensed pressure by a pressure-sensitive fabric and generate an alert based upon the electrical image representation.

FIG. 6 illustrates an example load testing result on a pressure-sensitive fabric configured in accordance with various embodiments.

FIGS. 7A and 7B illustrate example results of load testing prior to and subsequent to washing a pressure-sensitive fabric.

FIG. 8 is an example computing component that may be used to implement various features of embodiments described in the present disclosure.

The figures are not exhaustive and do not limit the present disclosure to the precise form disclosed.

### DETAILED DESCRIPTION

As alluded to above, the prevention of conditions such as pressure ulcers, skin damage, bone damage, tissue damage, etc. that can result from prolonged pressure on one or more body parts is preferable to treating the condition post-occurrence. Accordingly, monitoring the position of a patient's body is imperative so that the patient's body can be moved or shifted to relieve pressure, thereby avoiding such conditions.

Various embodiments described in the present disclosure are directed to a carbon nanotube-based, pressure-sensitive fabric that can be applied to or used to manufacture materials and/or apparatuses that come in contact with some load or pressure-inducing entity, such as a patient's body. For example, pressure-sensitive fabrics contemplated herein may be applied to bed mattresses, wheel chair cushions, hospital garments or other clothing, and the like. Such pressure-sensitive fabrics can be fabricated by integrating piezoresistive, multi-walled carbon nanotube (MWCNT)-latex thin films with flexible fabric.

Distributed pressure sensing in accordance with various embodiments described in the present disclosure can be achieved by utilizing electrical impedance tomography (EIT) to generate an image representative of one or more areas where a patient's body is applying pressure to the pressure-sensitive fabric. In particular, a pressure map indicative of pressure being exerted on the pressure-sensitive fabric is generated. EIT can be used to continuously map and calculate changes in electrical resistance across the entire area of the pressure-sensitive fabric. The changes in electric resistance or impedance are indicative of the pressure being applied to the pressure-sensitive fabric. Based on this mapping, a digital image can be generated, where contours representative of the impedance changes are correlated to one or more portions of a patient's body contacting the pressure-sensitive fabric. Evidence of prolonged pressure determined from the generated image can be used to trigger a notification or alarm that the patient should move or be moved.

Measuring distributed pressure across a given area usually depend upon some implementation of devices whose electrical properties can change in response to different magnitudes of applied strain. In order to acquire a pressure distribution for a given area, the changes in electrical signals at different spots or points where pressure is measured must be integrated with one another. This can involve instrumenting a dense network of sensors, and embedding the dense network of sensors within a fabric in order to obtain a high resolution map of applied pressure distributions.

Sensors typically used in the above-mentioned approaches are discrete sensors, e.g., strain gauges, fiber optics, etc. However, strain gauges, fiber optics, and similar discrete sensors are often bulky and difficult to integrate into a fabric. Even if integration of such sensors is possible, the result is a rigid fabric that can be invasive, and can often cause user discomfort. Furthermore, the need for instrumenting a dense network of sensors adds to the cost of fabrication, and complicates data acquisition. Further still, because discrete sensors are used, the reliability of distributed two-dimensional pressure is compromised, and error is introduced when attempting to interpolate data using only a limited number of discrete points of measurement.

Pressure sensors made with nanostructured or polymeric materials have been proposed to address the above-noted deficiencies associated with strain gauges, fiber optics, and the like. Flexible materials, such as micro-fluids (e.g., polydimethylsiloxane (PDMS) and polymethylmethacrylate (PMMA)) can be used as fillers in capacitive structures, which can generate capacitance changes once being deformed under pressure.

However, devices such as skin patches that are manufactured from the above pressure sensors are based upon inter-locking nano-fibers and polymers, and their fabrication can be cumbersome and expensive, making them unsuitable to be scaled up for use in practical applications. Furthermore, direct skin applications tend to be uncomfortable for the user, and despite being flexible, these materials have undesirably high sensitivity, and are highly reversible under fast loading.

As alluded to above, manufacturing distributed carbon nanotube-based, pressure-sensitive fabrics in accordance with various embodiments can be achieved by integrating a multi-walled carbon nanotube (MWCNT)-latex thin film with a fabric, and forming a flexible and self-sensing/pressure-sensitive fabric that can be put on chairs or beds, integrated into clothing, garment, or any wearable fabric, etc. When a patient or other subject sits or lies on the pressure-sensitive fabric, the pressure-sensitive fabric (which is flexible) will deform to an extent commensurate with the pressure applied by the weight of the patient or subject.

A MWCNT-latex thin film is electromechanically sensitive (as will be described below). Its electrical resistance changes in tandem with loaded strains. The electrical resistance of a pressure-sensitive fabric can be measured via electrodes, and the change in resistance across an entire sensing area of the pressure-sensitive fabric can be continuously mapped and calculated using the aforementioned EIT spatial conductivity mapping technique, where the electrical resistance of the pressure-sensitive fabric can be estimated throughout the area of the pressure-sensitive fabric. Because electrical resistance can be pre-calibrated to some external stimulus (e.g., pressure), a resulting electrical image generated based upon the electrical resistance corresponds directly to a pressure map. In this way, sitting, lying, or other positions creating pressure on the MWCNT-latex thin film can be readily visualized by referring to the resistance change contours which are indicative of the magnitude and location of the pressure exerted by the body. It should be noted that based on the selection of raw materials used during sensor development, a pressure-sensitive fabric can be encoded to sense and identify different parameters.

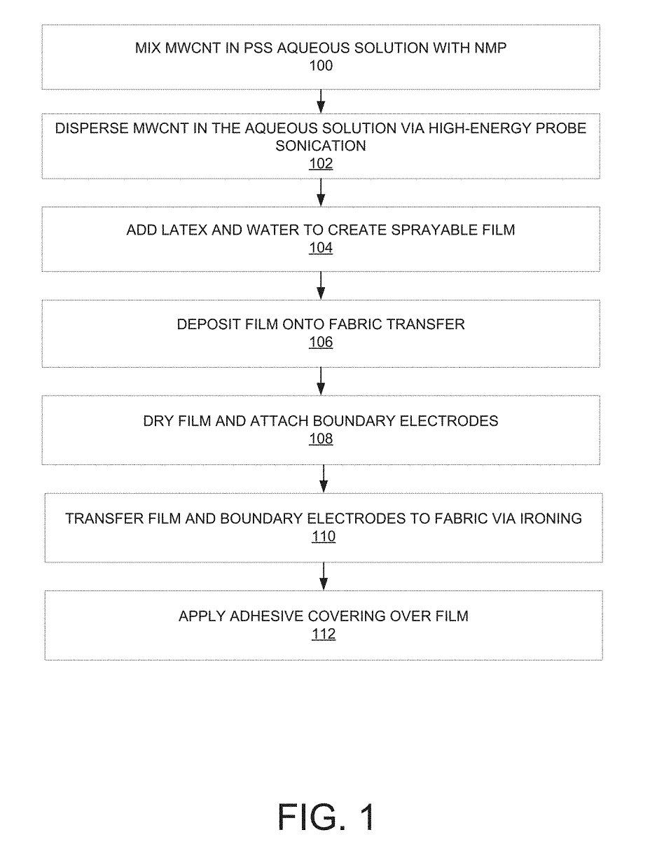

FIG. 1 illustrates various operations that can be performed to fabricate MWCNT-latex thin films using spray-fabrication techniques and apply the MWCNT-latex thin films to fabrics to create a pressure-sensitive fabric. For example, at operation 100, MWCNTs can be mixed with a 2 percent by weight (wt %) poly(sodium 4-styrenesulfonate) (PSS) aqueous solution having dilute amounts of N-methyl-2-pyrrolindinone (NMP). At operation 102, the MWCNT mixture can be subjected to 60 minutes of high-energy probe sonication (3 mm tip, 150 W, 22 kHz) for dispersing the MWCNTs. Once a well dispersed MWCNT/PSS-NMP solution is obtained, at operation 104, latex and water are added to the MWCNT/PSS-NMP solution to create a sprayable film. In one embodiment, a latex solution and deionized (DI) water are added in to obtain a sprayable ink. In one embodiment, the latex solution may be a 35 wt % Kynar Aquatec latex solution, and 3.5 wt % 18 MΩ DI water may be added.

At operation 106, the sprayable ink can be deposited onto a fabric transfer. For example, a 1 wt % thin film can be sprayed onto a fabric transfer. Spraying can be performed by holding a Paasche airbrush −30 cm perpendicular to the fabric transfer and by moving at a constant speed, although other methods and/or mechanisms known to those of ordinary skill in the art are contemplated. It should be noted that the dimensions of the films can be customized easily by changing the size of the fabric transfers. At operation 108, the MWCNT-latex film can be dried (e.g., air-dried for approximately 4 hours), after which electrodes can be attached along the boundaries of the film. This creates a thin film that is characterized by electrical properties which responds to applied strains, deformation, and/or pressure. If other sensing functionalities are desired, then the raw materials can be replaced with other more suitable materials that are sensitive to the stimulus or stimuli of interest.

It should be noted that the term “electrode” is intended to mean any conductive material, for example threads coated in a conductive solution, but other types and/or forms of electrodes are contemplated in accordance with other embodiments. For example, electrodes can be made of a variety of different electrically conductive materials and may be alloys or pure metals such as copper, gold, platinum, steel, silver, silver chloride, and alloys thereof. Further, the electrode may be comprised of a non-metal that is electrically conductive such as a silicon-based material used in connection with microcircuits. Electrodes may be used to deliver electrical current continuously or to deliver pulses.

At operation 110, the MWCNT-latex thin film along with the boundary electrodes may be transferred to a fabric by ironing the MWCNT-latex thin film onto the fabric. In one embodiment, the fabric on which the MWCNT-latex thin film is transferred may be a 100% polyester woven fabric material, although other fabrics may be utilized depending on the desired characteristics of the resulting pressure-sensitive fabric, e.g., greater pliability, desired water resistance, bonding characteristics, etc. At operation 112, an adhesive covering is applied over the MWCNT-latex thin film. Accordingly, a strong and integral sandwiched structure may be formed. It should be noted that in some embodiments, the MWCNT-latex thin films can be sprayed directly onto fabrics to create the distributed pressure-sensitive fabric as well. Example MWCNT-latex thin film production and use as distributed pressure sensors are described in “Spray-coated carbon nanotube-latex strain sensors” by Long Wang et al., Sci. Lett. J. 5 (234) (2016) and “Distributed Pressure Sensing using Carbon Nanotube Fabrics” by Long Wang et al., IEEE Sensors Journal, Vol. 16 Issue 12, 4663-4664, June 2016, both of which are incorporated herein by reference in their entirety.

It should be noted that the above-described method is one example of how pressure-sensitive fabric may be manufactured. Other methods of fabrication may include, drying, printing (e.g., inkjet printing), spin-coating, or filtering (through a membrane filter) the MWCNT-based solution onto a substrate. The substrate may be a fabric or other appropriate substrate, e.g., paper, plastic, polymer sheets or thin films, membranes, etc. so long as the substrate is flexible and can deform due to pressure.

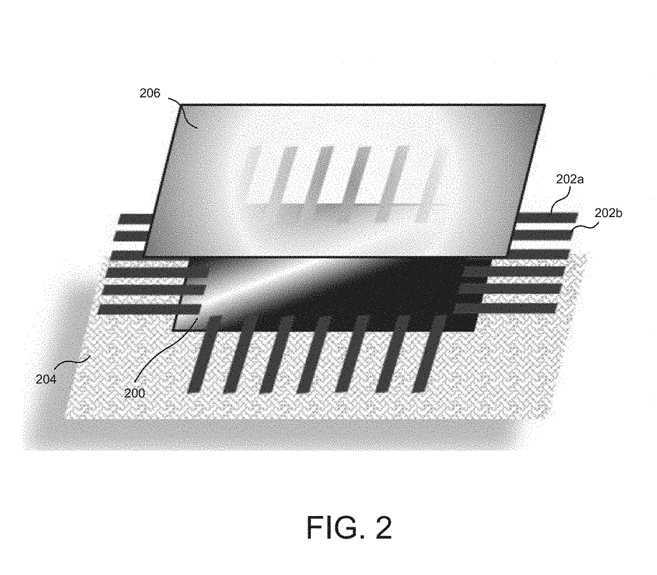

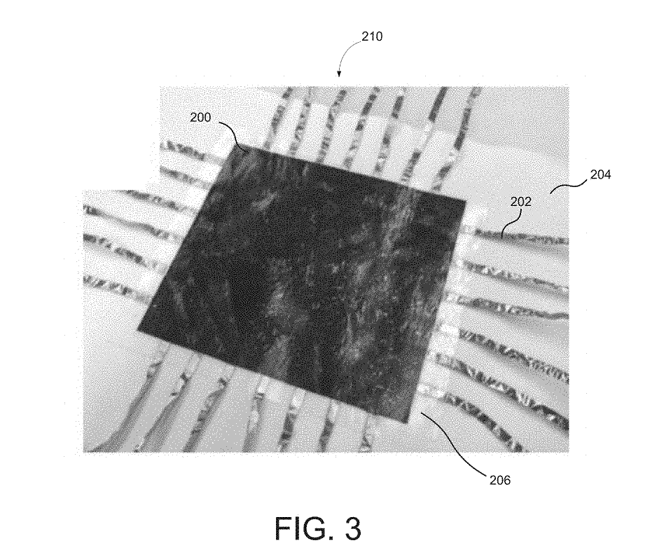

FIG. 2 is an example schematic representation of the component parts of a pressure-sensitive fabric made in accordance with various embodiments, for example, a pressure-sensitive fabric manufactured in accordance with the method illustrated in FIG. 1 and described above. FIG. 3 illustrates a completed pressure-sensitive fabric 210 made in accordance with various embodiments. FIGS. 2 and 3 illustrate a MWCNT-latex thin film 200 that may have been deposited onto a transfer fabric or deposited directly onto a fabric 204. In this example, 26 electrodes (e.g., electrodes 202a, 202b, etc.) are attached to the boundary of MWCNT-latex thin film 200. Although MWCNT-latex thin film 200 is illustrated as being approximately rectangular- or square-shaped, MWCNT-latex thin film 200 may take on any shape appropriate for the application in which it is to be used. Additionally, the size of MWCNT-latex thin film 200 may vary, as can the size, type, and shape(s) of the attached electrodes.

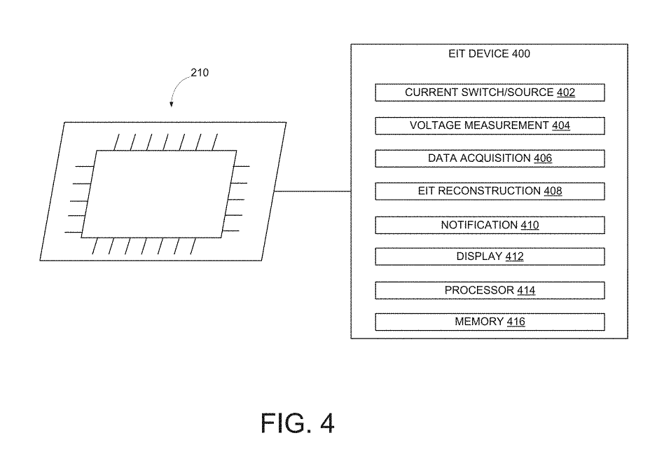

Adhesive covering 206 may be deposited onto or over MWCNT-latex film 200. It should be noted that in the example illustrated in FIGS. 2 and 3, adhesive covering 206 is configured to cover the entirety of MWCNT-latex thin film 200, while leaving at least a portion of each of the attached electrodes exposed to allow an electrical connection to be made to an EIT device, e.g., EIT device 400 illustrated in FIG. 4 and described in greater detail below. It should be noted that a garment, for example, may be made up of a plurality of pressure-sensitive fabric pieces or sections, or a garment may be made out of a single piece of pressure-sensitive fabric. The electrodes populating the boundary of the pressure-sensitive fabric define a region in which pressure can be sensed. The electrodes/electrode wiring may be fed to a multiplexer, creating what are essentially, different channels in which, e.g., hardware can be used to selectively choose to either inject current or interrogate (measure).

As previously mentioned, distributed pressure sensing in accordance with various embodiments can be achieved by utilizing EIT imaging to generate an image representative of one or more areas where a patient's body is applying pressure to the pressure-sensitive fabric. FIG. 4 illustrates a schematic representation of a pressure-sensitive fabric system in accordance with various embodiments.

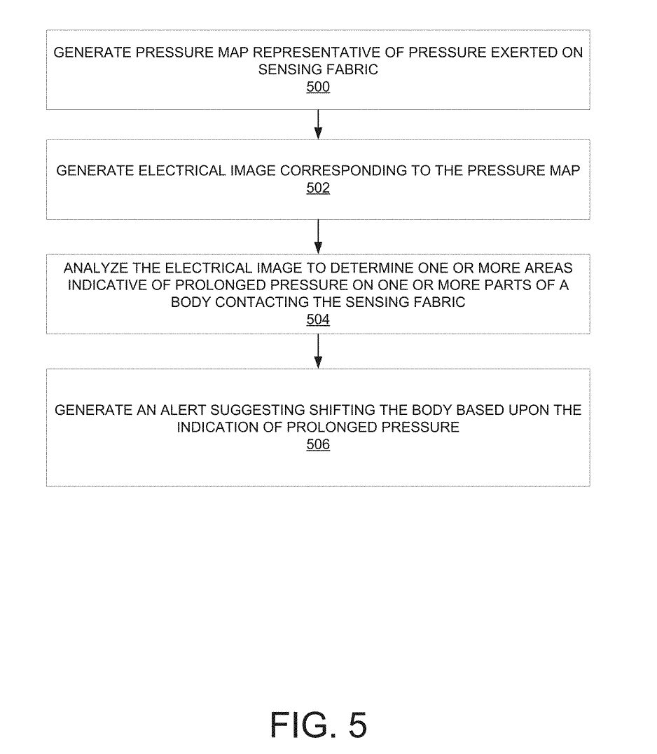

As illustrated in FIG. 4, a pressure-sensitive fabric 210 may be electrically connected to an EIT device 400. The EIT device 400 applies an EIT imaging technique to the pressure-sensitive fabric 210 to determine one or more areas of pressure experienced by pressure-sensitive fabric 210. FIG. 5 is a flow chart illustrating example operations that can be performed to effectuate distributed sensing in accordance with various embodiments, and will be described in conjunction with FIG. 4.

Generally, EIT is a tomography technique that seeks to reconstruct the spatial distribution of resistance and impedance of a conductive body based upon applied electrical input and measured output signals along the bodies boundary(ies). Accordingly, EIT uses data regarding, e.g., input current and boundary voltage measurements to solve an inverse problem in order to reconstruct the resistivity distribution of the conductive body. In the present disclosure, EIT can be used to reconstruct the resistivity distribution of the pressure-sensitive fabric, including when pressure is being applied to the pressure-sensitive fabric.

Using Laplace's 2D equation,

∇·[σ(x,y)∇v(x,y)]=0 (1)

the body's boundary potential or voltage distribution (v) can be calculated given a known electrical current source/sink and the body's conductivity distribution (α) represented by a Cartesian coordinate system. Equation (1) refers to a specific case in which electrical current is neither supplied nor generated within a 2D body, Ω. No discrete electrodes are defined in this continuum model. Instead, electrical current is defined as a continuous current function along the body's boundary, ∂Ω.

Given the complexity of the EIT problem, and the inability to apply a continuous boundary current function to Ω, the forward problem is often solved using a discretized weak formulation of Laplace's equation in conjunction with the finite element method (FEM). Equation (2) shows the weak form of Laplace's equation:

∫∫_{Ω}∇φ∇vdxdy=0 (2)

where φ is the shape function of the voltage at the electrodes. FEM and discretization allows the incorporation of discrete boundary electrodes, which is more practical than applying a continuous boundary current function. In addition, each FEM element assumes a constant value of conductivity or resistivity.

While the forward problem can be solved using Equation (2), EIT involves solving the “inverse problem.” That is, given a physical 2D body, Ω, with a set of discrete boundary electrodes, a direct current (DC) can be applied at one electrode, and another electrode can be set as ground. The potential drops or voltage differences at the remaining pairs of boundary electrodes (Vₑₓₚ) may then be measured. Referring back to FIG. 2, for example, current may be applied to electrode 202a, electrode 202b may be grounded, and voltage can be measured at the remaining electrodes along the boundary of MWCNT-latex thin film 200.

Here, σ(x,y) is unknown. Thus, solving the inverse problem begins by assuming a conductivity distribution so that the forward problem can be executed to determine the predicated boundary voltages (vₙᵤₘ). In accordance with various embodiments of the present disclosure, a Gauss-Newton iterative process can be implemented for updating the conductivity distribution of the body until the error difference (e) between experimental and predicted voltages is below a certain threshold, upon which the spatial resistivity distribution of the pressure-sensitive fabric can be estimated and output. Equation (3) is as follows:

e=//vₑₓₚ−vₙᵤₘ∥²≦0.05% (3)

It should be noted that EIT reconstruction is an ill-posed inverse problem, which in some embodiments, may include some prior regularization information in the reconstruction process (described in greater detail below).

An example EIT algorithm that may be utilized in accordance with various embodiments is as follows.

In some embodiments, user input to the EIT algorithm may include the following: L=Length of the specimen in millimeter; B=Breadth of the specimen in millimeter; Nₓ=Number of elements along the length of the specimen; N_{y}=Number of elements along the breadth of the specimen; and curr_mag=Magnitude of DC current used for EIT testing in ampere. Moreover, a current injection and measurement pattern may be specified via, e.g., an Excel file or other appropriate mechanism.

In some embodiments, a “Finite Element Stiffness Matrix” may be calculated as follows. Based on the input parameters, the rectangular area specified by the user is discretized into finite number of quadrilateral elements for finite element calculation. A Create_Mesh subroutine may be implemented to discretize the geometry into quadrilateral elements. Based on inputs L, B, Nx, Ny, the following are output: nodes (node matrix which contains the X and Y coordinates of each node); elements (element matrix which contains nodal connectivity information of each quadrilateral element); electrode (electrode matrix which contains all the surface node numbers for each boundary electrodes); rn (number of nodes in the finite element domain); ce (number of nodes contained by each element); r_elec (number of electrodes); and c_elec (number of nodes contained by each electrode).

A total stiffness matrix (G) may comprise four small matrices as described in equation 4.

$$G = [ \begin{matrix}{A}_{M} + {A}_{Z} & {A}_{V} \\ {A}_{V}^{T} & {A}_{D}\end{matrix} ] \qquad ( 4 )$$

A subroutine Create_AM_Q may be utilized to evaluate the A_{M} matrix, where inputs to the subroutine may be rn, re, elements, nodes, initial conductivity distribution (sigma), r_elec, c_elec, elec_impd, and electrodes, and wherein the output may be the A_{M} matrix. The A_{z}, A_{y}, and Aₓ matrices may also be evaluated accordingly in the main body of the program.

A subroutine Assemble A can be used to assemble the A_{M}, A_{z}, Aᵥ, and A_{d} matrices to create final G matrix.

Current vectors may be formulated. A current vector (I) is formulated for each injection pattern and used for evaluating nodal voltage values. A subroutine active elec may be used to create a logical vector array for marking the measurement electrodes (1 at the measurement electrodes and 0 for the excitation and ground electrodes). The input to the subroutine may be curr_inj_pat such that a logical vector for each injection pattern assembled in a matrix “act_elec” is output.

Another subroutine diff_meas may be used to mark the injection electrode as 1 and ground electrode as −1 for future calculations, where the subroutine input may be r_elec, and the output is a vector with 1 and −1 assembled in a matrix “C”.

A subroutine Voltage_Calc may be used to obtain nodal voltage distribution, where a linear system of equation is obtained from a finite element formulation. Inputs to this subroutine can include curr_inj_pat, rn, r_elec, curr_mag, G, act_elec, C. The resulting output may be the difference between the measurement electrode pairs for each injection pattern (elec V) and nodal voltage distribution (node voltage).

The aforementioned inverse problem refers to a Jacobian matrix that is calculated based on the results obtained from the execution of the forward problem. A Sensitivity subroutine can be used to evaluate the Jacobian matrix for the inverse algorithm. This Jacobian matrix may be used to store information regarding how one measurement is influenced by a little perturbation in conductivity of each element in the finite element domain. Inputs to this subroutine can include re, elements, rn, nodes, r_elec, A_{V}, A_{D}, curr_inj_pat, Aₛ, act_elec, node voltage, C, where the output may be the Jacobian matrix (Jₒ).

Additionally, a regularization matrix may be calculated from the Jacobian matrix calculated above. In particular, a Regularization subroutine may be used to achieve the aforementioned image reconstruction. That is, the EIT inverse problem is an “μl-posed problem” as a big change in conductivity distribution can result in a very small change in boundary voltage distribution. Proper regularization scheme may be adopted to make the problem “well-posed” so that an accurate solution can be achieved. In accordance with one embodiment, Tikhonov regularization can be used for the image reconstruction. Inputs to this subroutine may be Jₒ and re, with the output being a regularization matrix (Reg).

A boundary voltage distribution obtained from, e.g., from laboratory testing may be loaded and stored in a variable, Vₘ.

As described above, a Gauss-Newton iterative process can be implemented for updating the conductivity distribution of the body until the error difference (e) between experimental and predicted voltages is below a certain threshold, upon which the spatial resistivity distribution of the pressure-sensitive fabric sensor can be estimated and output. Accordingly, a Gauss-Newton iterative inverse algorithm may be utilized to minimize the error norm between the measured (Vₘ) and computed set of boundary voltages (V_{c}). The steps of iterative Gauss-Newton algorithm are described below.

Referring back to equation 3, vector (e) may be calculated by evaluating the difference between Vₘ and V_{c} and the norm of the error vector is calculated.

Error ratio (eᵣ) can be estimated by evaluating the ratio between the norm of e and the norm of Vₘ.

$${e}_{r} = \frac{{‖ e ‖}^{2}}{{‖ {V}_{m} ‖}^{2}} \qquad ( 5 )$$

It is checked if eᵣ satisfies the specified threshold criteria.

eᵣ≦0.05% (6)

If it does not satisfy the specified criteria, the conductivity distribution is updated by using equation 6.

If it does not satisfy the specified criteria, the conductivity distribution is updated by using equation 7, where Jᵢ is the Jacobian matrix evaluated at i^{th} iteration step, a is the regularization parameter, σᵢ₊₁ and σᵢ are the conductivity distributions at i+1^{th} and i^{th} iteration step respectively while σ₀ is the initially assumed background conductivity distribution.

σᵢ₊₁=σᵢ+(Jᵢ^{T}Jᵢ+α²R^{T}R)⁻¹(Jᵢ^{T}V_{c}−Vₘ)+α²R^{T}R(σᵢ−σₒ)) (7)

The iteration runs as long as the specified criteria is not satisfied.

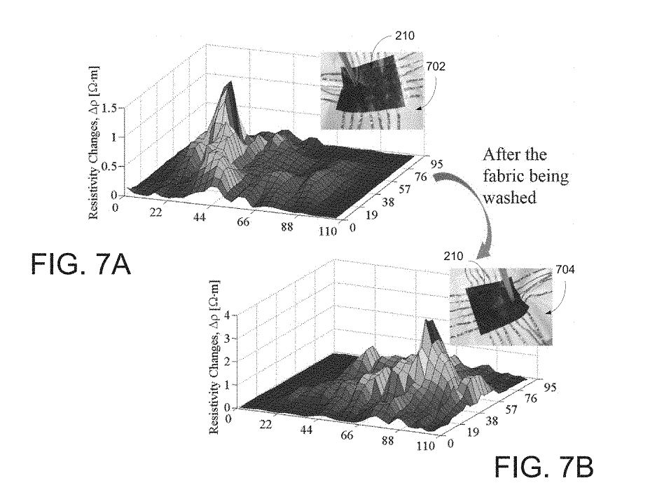

A final conductivity distribution can be plotted after the algorithm converges to the specified threshold limit. To accomplish this, a plot conductivity subroutine may be utilized to plot the final conductivity distribution in a 2D plane with color contour. Inputs to this subroutine may be nodes, elements, sigma, with an output being a final conductivity distribution plot. In some embodiments, the final conductivity distribution play may be a rainbow color plot with a corresponding color bar, examples of which are illustrated in FIGS. 7A and 7B described below.

Referring back to FIG. 4, EIT device 400 is shown to comprise a current switch and source 402. Current switch and source 402 may be used to inject current into the electrodes of pressure-sensitive fabric sensor 210. A current switch is utilized in order to switch between respective electrode pairs such that current can be injected into two electrodes at a time, while (as described above) voltage is measured at the remaining boundary electrodes. Those of ordinary skill in the art would be aware of appropriate switching devices/multiplexers that can be utilized to implement this functionality.

EIT device 400 may also include a voltage measurement component 404 to perform the aforementioned measurement of voltage across the boundary electrodes. It should be noted that in some embodiments, the voltage measurements may be amplified and/or filtered to handle, e.g., noise, filter out certain frequencies, etc. Data acquisition component 406 can obtain the voltage measurements for storage, e.g., in memory unit 416.

Utilizing the obtained voltage measurements, EIT reconstruction component 408 implements the aforementioned EIT algorithm in order to reconstruct an image of the pressure-sensitive fabric 210 indicating any pressure points (location and magnitude) that have been sensed. That is, and referring to FIG. 5, EIT reconstruction component 408 generates a pressure map representative of pressure exerted on pressure-sensitive fabric 210 (operation 500), and generates an electrical or digital image corresponding to the pressure map (operation 502). The generated electrical image may be displayed on display 412.

Notification component 410, alone or in conjunction with processor 414, can analyze the electrical image generated by EIT reconstruction component 408 to determine whether one or more areas of pressure-sensitive fabric 210 has experienced prolonged pressure as a result of a body contacting one or more parts pressure-sensitive fabric 210 (operation 504).

Notification component 410 and/or processor 414 may compare baseline electrical images representative of pressure-sensitive fabric 210 in “non-pressurized” states with one or more subsequent electrical images over some period of time. If the electrical image indicates the existence of one or more pressure points on pressure-sensitive fabric 210 for some period of time meeting a threshold, notification component 410 may generate an alert suggesting shifting the body (operation 506). The threshold may vary depending on various factors including, e.g., the particular medical condition of the patient being monitored, the age of the patient, etc. Those of ordinary skill in the art would be aware of relevant thresholds to be used for comparison.

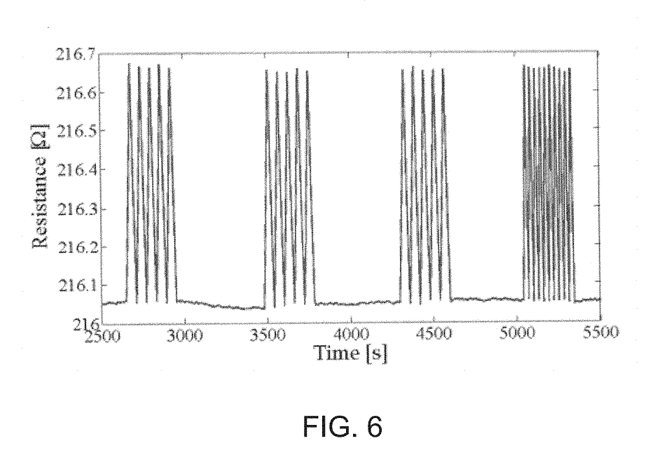

FIG. 6 illustrates an example result of load testing on a pressure-sensitive fabric configured in accordance with various embodiments. In particular, FIG. 6 illustrates a representative time history of a pressure-sensitive fabric subjected to multiple sets of tensile cyclic loads up to 10,000 micro strain (μE). It can be appreciated that the resistance of the pressure-sensitive fabric increases in tandem with increasing pressure, and the correlation between applied pressure and resistance change is linear.

Pressure-sensitive fabrics or materials in accordance with various embodiments are capable of continuously mapping pressure distribution contours, are non-invasiveness, scalable, and hand-washable. For example, as described above, MWCNT-latex thin films can be spray fabricated, which is low-cost, simple, and efficient. Additionally, the thin films can be easily scaled up to larger sizes, and are suitable for large-scale manufacturing. It should also be noted that the spray fabrication process can be automatically accomplished via controlling a robotic spray arm, which can simply manufacturing, as well as achieve uniformity in the sensors. Furthermore, the ironing process that completes fabrication of a pressure-sensitive fabric is a mature technique in the clothing industry.

A pressure-sensitive fabric in the form of, e.g., bedding sheets, clothing, etc. is flexible. To a user/patient, garments and sheets made of or including pressure-sensitive fabrics in accordance with various embodiments feel the same as non-pressure-sensitive fabrics. Spraying MWCNT-latex thin film directly onto fabrics can, in some instances, increase their rigidity, but this can be mitigated. For example, a material other than latex may be used, where this material is similar to the fabric itself. As another example, a thinner coating of the MWCNT-latex thin film can be applied. Moreover, due to its flexibility, the pressure-sensitive fabric is able to softly conform to the human body, providing a comfortable and noninvasive user experience.

Further still, the pressure-sensitive fabric is capable of measuring strains across its entire area. By analyzing the electrical impedance changes of the pressure-sensitive fabric, continuous mapping of pressure distribution can be acquired with favorable sensitivity in measuring pressure magnitudes (0.021 Ω·Pa⁻¹) and remarkable accuracy determining loading locations.

A pressure-sensitive fabric configured in accordance with various embodiments proves to be hand-washable. The pressure sensing performance of the pressure-sensitive fabric is maintained after being hand-washed in water for, e.g., 20 minutes, during which, it can be folded and twisted. It should be noted that this feature or property of the pressure-sensitive fabric cannot be easily attained using the previously mentioned electrical devices and/or nano- and polymeric material-based pressure sensors.

FIGS. 7A and 7B illustrate example pressure distributions that were mapped using EIT reconstruction described above of a pressure-sensitive fabric 210 prior to washing and post-washing. Prior to washing, FIG. 7A illustrates a correspondence between the location of the application of pressure (see inset 702) on pressure-sensitive fabric 210 and a localized increase in resistivity at/near the area where pressure was applied. FIG. 7B illustrates that after pressure-sensitive fabric 210 was washed (in this case 10 minutes of washing, and 3 hours of air-drying) and re-calibrated, the correspondence between the location of the application of pressure (see inset 704) on pressure-sensitive fabric 210 and a localized increase in resistivity at/near the area where pressure was applied remains.

It should be noted that various embodiments of the technology disclosed in the present example are described in the context of preventing harmful, pressure-related medical conditions. However, various embodiments can also be utilized in other context, such as sensing how humans move, as well as where and to what extent humans are applying pressure to their surroundings. Moreover, other external stimuli or human physiological responses that can be monitored include temperature, pH, and humidity, among others.

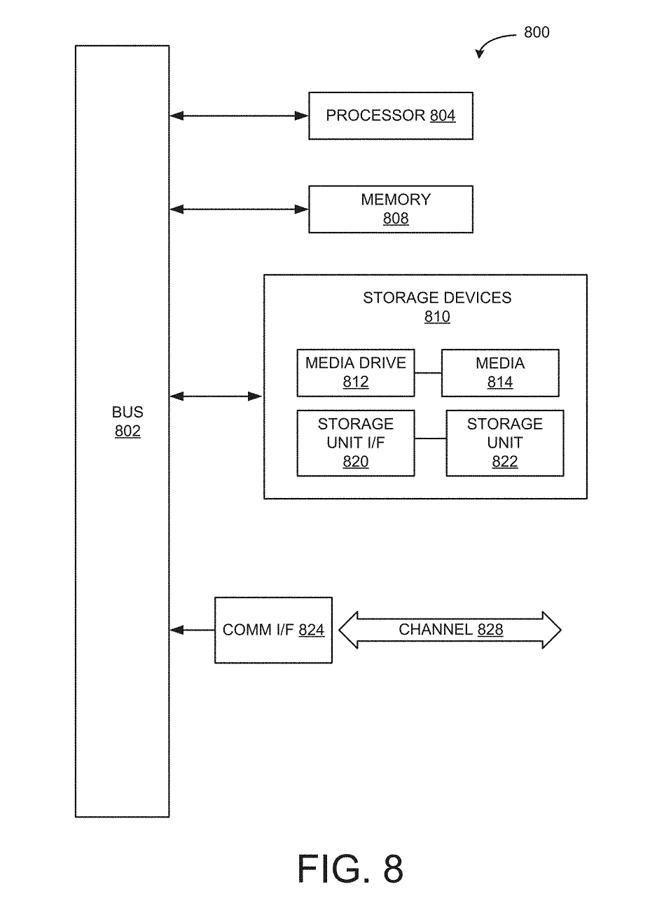

As used herein, the term component might describe a given unit of functionality that can be performed in accordance with one or more embodiments of the present application. As used herein, a component might be implemented utilizing any form of hardware, software, or a combination thereof. For example, one or more processors, controllers, ASICs, PLAs, PALs, CPLDs, FPGAs, logical components, software routines or other mechanisms might be implemented to make up a component. In implementation, the various components described herein might be implemented as discrete components or the functions and features described can be shared in part or in total among one or more components. In other words, as would be apparent to one of ordinary skill in the art after reading this description, the various features and functionality described herein may be implemented in any given application and can be implemented in one or more separate or shared components in various combinations and permutations. Even though various features or elements of functionality may be individually described or claimed as separate components, one of ordinary skill in the art will understand that these features and functionality can be shared among one or more common software and hardware elements, and such description shall not require or imply that separate hardware or software components are used to implement such features or functionality.

Where components or components of the application are implemented in whole or in part using software, in one embodiment, these software elements can be implemented to operate with a computing or processing component capable of carrying out the functionality described with respect thereto. One such example computing component is shown in FIG. 8. Various embodiments are described in terms of this example-computing component 800. After reading this description, it will become apparent to a person skilled in the relevant art how to implement the application using other computing components or architectures.

Referring now to FIG. 8, computing component 800 may represent, for example, computing or processing capabilities found within a self-adjusting display, desktop, laptop, notebook, and tablet computers; hand-held computing devices (tablets, PDA's, smart phones, cell phones, palmtops, etc.); workstations or other devices with displays; servers; or any other type of special-purpose or general-purpose computing devices as may be desirable or appropriate for a given application or environment. Computing component 800 might also represent computing capabilities embedded within or otherwise available to a given device. For example, a computing component might be found in other electronic devices such as, for example, portable computing devices, and other electronic devices that might include some form of processing capability.

Computing component 800 might include, for example, one or more processors, controllers, control components, or other processing devices, such as a processor 414, and/or any one or more of the components making up EIT device 400. Processor 804 might be implemented using a general-purpose or special-purpose processing engine such as, for example, a microprocessor, controller, or other control logic. In the illustrated example, processor 804 is connected to a bus 802, although any communication medium can be used to facilitate interaction with other components of computing component 800 or to communicate externally.

Computing component 800 might also include one or more memory components, simply referred to herein as main memory 808. For example, preferably random access memory (RAM) or other dynamic memory, might be used for storing information and instructions to be executed by processor 804. Main memory 808 might also be used for storing temporary variables or other intermediate information during execution of instructions to be executed by processor 804. Computing component 800 might likewise include a read only memory (“ROM”) or other static storage device coupled to bus 802 for storing static information and instructions for processor 804.

The computing component 800 might also include one or more various forms of information storage mechanism 810, which might include, for example, a media drive 812 and a storage unit interface 820. The media drive 812 might include a drive or other mechanism to support fixed or removable storage media 814. For example, a hard disk drive, a solid state drive, a magnetic tape drive, an optical disk drive, a compact disc (CD) or digital video disc (DVD) drive (R or RW), or other removable or fixed media drive might be provided. Accordingly, storage media 814 might include, for example, a hard disk, an integrated circuit assembly, magnetic tape, cartridge, optical disk, a CD or DVD, or other fixed or removable medium that is read by, written to or accessed by media drive 812. As these examples illustrate, the storage media 814 can include a computer usable storage medium having stored therein computer software or data.

In alternative embodiments, information storage mechanism 810 might include other similar instrumentalities for allowing computer programs or other instructions or data to be loaded into computing component 800. Such instrumentalities might include, for example, a fixed or removable storage unit 822 and an interface 820. Examples of such storage units 822 and interfaces 820 can include a program cartridge and cartridge interface, a removable memory (for example, a flash memory or other removable memory component) and memory slot, a PCMCIA slot and card, and other fixed or removable storage units 822 and interfaces 820 that allow software and data to be transferred from the storage unit 822 to computing component 800.

Computing component 800 might also include a communications interface 824. Communications interface 824 might be used to allow software and data to be transferred between computing component 800 and external devices. Examples of communications interface 824 might include a modem or softmodem, a network interface (such as an Ethernet, network interface card, WiMedia, IEEE 802.XX or other interface), a communications port (such as for example, a USB port, IR port, RS232 port Bluetooth® interface, or other port), or other communications interface. Software and data transferred via communications interface 824 might typically be carried on signals, which can be electronic, electromagnetic (which includes optical) or other signals capable of being exchanged by a given communications interface 824. These signals might be provided to communications interface 824 via a channel 828. This channel 828 might carry signals and might be implemented using a wired or wireless communication medium. Some examples of a channel might include a phone line, a cellular link, an RF link, an optical link, a network interface, a local or wide area network, and other wired or wireless communications channels.

In this document, the terms “computer program medium” and “computer usable medium” are used to generally refer to transitory or non-transitory media such as, for example, memory 808, storage unit 820, media 814, and channel 828. These and other various forms of computer program media or computer usable media may be involved in carrying one or more sequences of one or more instructions to a processing device for execution. Such instructions embodied on the medium, are generally referred to as “computer program code” or a “computer program product” (which may be grouped in the form of computer programs or other groupings). When executed, such instructions might enable the computing component 800 to perform features or functions of the present application as discussed herein.

Although described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the application, whether or not such embodiments are described and whether or not such features are presented as being a part of a described embodiment. Thus, the breadth and scope of the present application should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. The use of the term “component” does not imply that the aspects or functionality described or claimed as part of the component are all configured in a common package. Indeed, any or all of the various aspects of a component, whether control logic or other components, can be combined in a single package or separately maintained and can further be distributed in multiple groupings or packages or across multiple locations.

Additionally, the various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

What is claimed is:

1. A method comprising:
mixing multi-walled carbon nanotubes in an aqueous solution;
dispersing the multi-walled carbon nanotubes in the aqueous solution;
adding latex and water to create a sprayable multi-walled carbon nanotube film;
depositing the multi-walled carbon nanotube film on a fabric transfer;
drying the multi-walled carbon nanotube film;
attaching boundary electrodes about a boundary of the multi-walled carbon nanotube film;
transferring the multi-walled carbon nanotube film and the boundary electrodes to a fabric; and
applying an adhesive covering over the multi-walled carbon nanotube film.

2. The method of claim 1, wherein the aqueous solution comprises a 2 weight percent poly (sodium 4-styrenesulfonate) and N-methyl 2-pyrrolindinone aqueous solution.

3. The method of claim 1, wherein the dispersing of the multi-walled carbon nanotubes comprises high-energy sonication.

4. The method of claim 1, wherein the water comprises deionized water.

5. The method of claim 1, wherein the transferring of the multi-walled carbon nanotube film to the fabric comprises ironing the multi-walled carbon nanotube film onto the fabric.

6. A method comprising:
generating a pressure map representative of pressure exerted on a sensing fabric;
generating an electrical image corresponding to the pressure map;
analyzing the electrical image to determine one or more areas indicative of prolonged pressure on one or more parts of a body contacting the sensing fabric; and
generating an alert suggesting shifting the body based upon the indication of prolonged pressure.

7. The method of claim 6 further comprising, injecting current into two of a plurality of boundary electrodes comprising the sensing fabric.

8. The method of claim 7 further comprising, measuring voltage at remaining ones of the plurality of boundary electrodes.

9. The method of claim 8 further comprising, repeating the injection of the current into alternative pairs of the plurality of boundary electrodes while repeating the measurement of the voltage at the remaining ones of the plurality of boundary electrodes.

10. The method of claim 9, wherein the generation of the pressure map is based upon the voltage measurements at the plurality of boundary electrodes.

11. The method of claim 9, wherein the measured voltage is at least one of amplified and filtered.

12. The method of claim 1, wherein analyzation of the electrical image comprises applying an electrical impedance tomography algorithm to reconstruct an image of the sensing fabric indicating changes in impedance in the sensing fabric due to pressure from the one or more parts of the body on the sensing fabric.

13. A system comprising:
a fabric;
a multi-walled carbon nanotube thin film operatively attached to the fabric;
a plurality of electrodes operatively attached to a boundary of the multi-walled carbon nanotube thin film, wherein alternating pairs of the plurality of electrodes are injected with current, and voltage measurements are taken at remaining ones of the plurality of electrodes, such that a visual representation of points and magnitudes of pressure on the fabric is presented based upon the voltage measurements.

14. The system of claim 13, wherein the multi-walled carbon nanotube thin film comprises a multi-walled carbon nanotube-latex thin film.

15. The system of claim 13, wherein the fabric, the multi-walled carbon nanotube thin film, and the plurality of electrodes are washable.

16. The system of claim 13 further comprising, an adhesive layer covering the multi-walled carbon nanotube thin film such that the multi-walled carbon nanotube thin film is sandwiched between the fabric and the adhesive layer.

17. The system of claim 13 further comprising, an electrical impedance tomography device operatively connected to the plurality of electrodes.

18. The system of claim 17, wherein the electrical impedance tomography device comprises a current switch that injects the current into the alternating pairs of the plurality of electrodes.

19. The system of claim 17, wherein the electrical impedance tomography device comprises a display that displays the visual representation, the points and magnitudes of pressure on the fabric reflecting changes in impedance in the multi-walled carbon nanotube thin film.

20. The system of claim 17, wherein the electrical impedance tomography device comprises a notification component that generates one or more notifications or alarms upon the multi-walled carbon nanotube thin film sensing prolonged pressure thereon.
